(19) United States
(12) Patent Application Publication
(10) Pub. No.: US 2008/0234311 A1
(43) Pub. Date: Sep. 25, 2008

(54) IMMUNOASSAY FOR DETECTION AND QUANTIFICATION OF AMYLOID-beta PEPTIDES

(76) Inventors: Jinhe Li, Long Grove, IL (US); Murali Gopalakrishnan, Libertyville, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/942,395

(22) Filed: Nov. 19, 2007

Related U.S. Application Data
(63) Continuation-in-part of application No. 11/936,432, filed on Nov. 7, 2007, now abandoned.
(60) Provisional application No. 60/857,895, filed on Nov. 8, 2006.

(51) Int. Cl. A61K 31/44; G01N 33/543; G01N 33/53; A61P 25/00; C07D 451/00; C12N 5/06

(52) U.S. Cl. 514/295; 436/547; 435/7.21; 436/518; 546/94; 435/375

(57) ABSTRACT

The present invention provides a method for the detection and quantification of Aβ₁₋₄₀ produced in native cell types and tissues. Also provided are assays and kits to determine the effect of compounds on the production of amyloid β peptides.

This application is a continuation-in-part of U.S. application Ser. No. 11/936,432 filed on Nov. 7, 2007, which claims priority on provisional application Ser. No. 60/857,895 filed on Nov. 8, 2006.

### TECHNICAL FIELD AND BACKGROUND

The present application relates to diagnostic methods for detection and quantification of amyloid-β peptides. More particularly, the application relates to an immunoassay for direct measurements of rodent amyloid-β peptides produced by native cell types and in tissues using small samples and reduced number of steps.

Alzheimer's disease (AD) is the most common form of dementia. AD patients undergo memory loss, general cognitive decline, impairment of judgment and problem solving, deterioration of language abilities, and eventually behavioral and personality changes and motor complication along with the dementia and end stage. There are two pathological markers of AD: senile plaques and neurofibrillary tangles (NFTs) (for a review, see Walsh and Selkoe, Neuron Vol. 44 pages 181-93, 2004). The plaques form in the neocortex, hippocampus, and amygdala of AD brains, the regions involved in learning and memory. Evidence from analysis of plaque structure and formation in AD and transgenic mice indicates that the plaques form by reversible aggregation of amyloid β peptides (Aβ) in clusters. On the other hand, NFTs are intraneuronal lesions of paired helical filaments made of hyperphosphorylated tau protein. Tangles are mostly found in the subiculum, cornu ammonis 1 region of the hippocampus, entorhinal cortex and neocortex.

Aβ is generated from amyloid precursor protein (APP) through sequential proteolytic cleavages (for a review, see Selkoe, J Clin Invest. 110:1375-81, 2002). Majority of APP is cleaved at α site by α-secretase, generating soluble APP (sAPPα) for which the function is not well understood. Only about 5% of APP is cleaved by β-secretase (also name BACE) at β-site, generating the C-terminal fragment of APP named CT99. CT99 is then cleaved by γ-secretases at amino acid 40 or 42, generating Aβ₁₋₄₀ or Aβ₁₋₄₂, respectively. There is considerable evidence indicating that Aβ, particularly the Aβ₁₋₄₂ is one of the major factors in AD pathogenesis (for a review, see Selkoe, J Clin Invest. Vol. 110 pages 1375-1381, 2002). Mutations around the α, β or γ-site of APP have been identified in familial AD, which result in increased Aβ production. Other mutations linked to familial AD include the ones in presenilin 1 and 2 genes (PS1 and PS2), which cause increased ratio of Aβ₁₋₄₀/Aβ₁₋₄₂. Transgenic animals expressing clinical mutant form of APP produced Aβ as well as plaques, and demonstrated deficit in cognitive and synaptic functions (Spires and Hyman, NeuroRx Vol. 2 pages 423-437, 2005). Passive and active immunization of APP transgenic animals against Aβ₁₋₄₂ reduced cognitive deficits. It has also been widely reported that Aβ₁₋₄₂ can cause toxicity in neuronal cells (for a review, see Selkoe, J Clin Invest. Vol. 110 pages 1375-1381, 2002). Collectively, these observations support the notion that accumulation of amyloid-β peptides in brain plays a central role in AD pathogenesis. Accordingly, a wide range of approaches aiming at reduction of Aβ has been taken in the search for novel AD therapies. It is hypothesized that reduction of Aβ levels by a range of approaches can have potential for disease modification in AD, which can prevent or reverse cognitive deficits. Results obtained from nicotine and other ligands interacting with acetylcholine neuronal nicotininc receptors (nAChR)suggest that agonist activating these receptors may modify AD pathological pathway by interfering either with the toxicity or the production of Aβ.

Currently available methods for measurement of Aβ peptides include immuno-precipitation and Western Blot assays requiring long and complicated procedures, involving large amount of samples, and detecting only over-expressed exogenous Aβ (Sun et al., JBS Vol. 278, pages 27688-27694, 2003). Other available methods include electroluminescence immunoassays which detect endogenous amyloid β in the central nervous system only; it involves a relatively fast procedure but requires restricted light and timing conditions (Best et al., JPET Vol. 313, pages 902-908, 2005).

Currently available enzyme-linked immunoassay involves the need of large samples (100 μL) and long and elaborate procedures (five steps, two days), and can detect only over-expressed mutant human APP in transfected cells, in tissues of transgenic mice, or in brain tissue of non-transgenic rodent. The overexpression of exogenous mutant APP is problematic, in particular because it may lead to a condition less physiological compared to that in AD brains, which in turn may not provide a physiological assessment of effect of agents that target Aβ production. (Sun et al., JBC Vol. 278 pages 27688-27694, 2003; Best et al., JPET Vol. 313 pages 902-908, 2005).

Therefore, it would be advantageous to provide a specific assay for the detection and quantification of amyloid β peptides in small samples and short amounts of time, without the need of overexpressing exogenous mutant APP, i.e. detection and quantification of Aβ peptides produced by rodent neuronal cell cultures. Furthermore, it would be advantageous to have methods that permit detection and quantification of Aβ peptides in samples other than brain tissue, for example plasma. The availability of such methods would facilitate the identification of novel compounds and mechanisms that lower endogenous Aβ peptide levels. As a whole, such methods could aid the identification of novel amyloid-targeted therapeutic approaches for Alzheimer's disease.

### BRIEF DESCRIPTION OF THE FIGURES

FIG. 1. (A) Scheme of the Assay for Detection of Mouse and Rat Aβ₁₋₄₀ and Aβ₁₋₄₂; (B) Specificity of the assay for rodent Aβ; (C) Sensitivity of the assay for detection of Aβ₁₋₄₀ and Aβ₁₋₄₂; (D) Results of the assay performed in a one step procedure. For (A-C) X axis represents concentration of Aβ detected and Y axis is optical density.

FIG. 2. (A) Aβ levels detected in conditioned medium obtained from rat primary cortical neurons after 4 days of culture; (B) Inhibition of Aβ₁₋₄₀ production in cortical neurons by inhibitor of gamma secretase L-685,458; (C): Aβ levels detected in mouse brain and plasma.

FIG. 3. Example of a high throughput assay for Screening Compounds that Modify Production of Rat Aβ₁₋₄₀.

FIG. 4. (A-E) Examples of compounds identified as α7 nicotinic receptor ligands that modify the production of Aβ₁₋₄₀ in rats

### DETAILED DESCRIPTION

The present application provides an immunoassay for detecting endogenous rodent amyloid-β peptides 1-40 and 1-42 amino acids (Aβ₁₋₄₀ and Aβ₁₋₄₂) produced in native cell types and tissues without the need for overexpressing exogenous amyloid precursor protein (APP), a method of using the same, and related articles of manufacture.

As stated above, most current assays measure Aβ production in transfected cells or transgenic mice over-expressing mutant human APP because the measurement of endogenous human Aβ has been difficult. This is due to the lack of a cell line producing high enough levels of Aβ. The assay described in the present application detects endogenous mouse/rat Aβ₁₋₄₀ and Aβ₁₋₄₂ in samples generated from neuronal cell cultures and animal tissues. This assay is specific for rodent Aβ and distinguishes Aβ₁₋₄₀ or Aβ₁₋₄₂ from each other.

In its principal embodiment the present application provides for a method for detecting the amount of rodent amyloid β peptides in a sample contacting the sample with a monoclonal antibody selective for N-temrinal of rodent Aβ for a time and under conditions to form complexes; contacting said complexes with a polyclonal anti-Aβ₁₋₄₀ or Aβ₁₋₄₂ antibody for a time and under conditions to form complexes; contacting said complexes with a secondary antibody linked to a detectable label capable of generating a measurable signal; incubating the mixture of the previous step for a time and under conditions to form complexes and the development of a measurable signal; and determining the amount of amyloid β peptide in the sample by detecting the signal generated. It is intended that the steps can be performed individually or combined in one single step.

The term “sample” or “test sample”, as used herein, includes biological samples which can be tested by the method of the present invention and include body fluids such as whole blood, serum, plasma, cerebrospinal fluid, urine, lymph fluids, and biological fluids such as cell culture supernatants. Any substances that can be adapted for testing with the reagents described herein and assay formats of the present invention are contemplated to be within the scope of the present invention.

It is intended that the rodent amyloid β peptide that can be detected and quantified by the method described in the present application can be Aβ₁₋₄₀ and/or Aβ₁₋₄₂. It is also intended that the sample that can be used for the method described in the present application be selected from the group comprising brain homogenates, whole blood, serum, plasma, cerebrospinal fluid, urine, lymph fluids, and cell culture supernatants.

Mouse and rat Aβ₁₋₄₀ and Aβ₁₋₄₂ are identical to each other, but are different from human Aβ(s) at three amino acids at the N-terminal of the peptides. In the present invention, an antibody against N-terminal domain of mouse/rat Aβ is used as capture antibody that captures various Aβ species in samples such as conditioned medium from a cell culture, cellular lysate, or animal tissues during an incubation over time. The mouse/rat Aβ₁₋₄₀ or Aβ₁₋₄₂ can be determined by binding of primary antibodies specific for either Aβ₁₋₄₀ or Aβ₁₋₄₂ C-termini, which can be detected by subsequent binding of a signal-generating secondary antibody against the primary antibody IgG followed by the detection of the signal.

A signal-generating secondary antibody means a compound that is capable of generating and generates a measurable signal detectable by external means (e.g., light, fluorescence, color), conjugated to an immunoreactive specific binding member, for example an antibody member of a specific binding pair. The immunoreactive specific binding member can be an antibody, an antigen, or an antibody/antigen complex that is capable of binding either to the polypeptide of interest as in a sandwich assay, to the capture reagent as in a competitive assay, or to the ancillary specific binding member as in an indirect assay. The commonly used labels for secondary antibody include horseradish peroxidase (HRP) that oxidize substrates into color molecules. Two substrates commonly used to detect HRP-conjugated antibodies are 4-chloro-1-naphthol (CN) and TMB (3,3′,5,5′-tetramethylbenzidene). TMB can be turned into blue color by HRP, which then turn to yellow and is stabilized by 0.2N H₂SO₄. The yellow color can be detected in a spectrophotometer at wavelength of 450 nM.

The method described in the present application can be performed in a total effective time of less than 5 hours, preferably 3 hours and, more preferably in 2 hours. It is contemplated that the sample size may vary from 1 μL to 25 μL, facilitating the number of samples that can be tested in one single experiment.

The present application also provides a method for screening a plurality of compounds for potential modification of the production of Aβ, and for identifying at least one compound, which specifically reduces the production of endogenous rodent amyloid β peptide in a primary neuron cell culture. It is contemplates that the method can be performed by following the steps of contacting a compounds or a plurality of compounds (agent(s)) with the rodent primary neuron cell culture; detecting the production of amyloid β peptide in the culture in the presence or absence of the agent(s) using the method described above, and comparing the amounts of detected amyloid β peptide in the presence or absence of the agent(s), wherein said comparison identifies the agent(s) as a compound that alters the production of amyloid β peptide, which can be either Aβ₁₋₄₀ and Aβ₁₋₄₂.

α7 nicotinic acetylcholine receptors (α7 nAChRs) belong to the family of acetylcholine-gated cation channels, which contains twelve subunits, α2-α10 and β2-β4 (for a review, see Dajas-Bailador and Wonnacott, Sciences Vol. 25, pages 317-324, 2004). These subunits are differentially expressed in the nervous system and combine to form nAChRs with a wide range of physiological and pharmacological profiles. Unlike heteromeric nAChRs (e.g. α4β2 or α3β4), the α7 nAChRs are homo-oligomeric receptors that exhibit distinct properties in terms of activation/desensitization kinetics, ionic (Ca2+ vs. Na+) selectivity (Seguela et. al. J. Neurosci. Vol. 13, pages 596-604, 1993), biochemical signaling and pharmacological selectivity (for recent reviews, see Hogg and Bertrand Current Drug Targets—CNS & Neurological Disorders Vol. 3, pages 123-130, 2004; Gotti and Clementi, Progress in neurobiology Vol. 74, pages 363-396, 2004). The nAChRs can be activated upon binding of their agonists, which can be either blocked by their antagonists or enhanced by positive allosteric modulators.

α7 nAChRs are expressed in several brain regions, especially localized at presynaptic and postsynaptic levels in the hippocampus and cerebral cortex, regions critical to synaptic plasticity underlying learning and memory. Presynaptic α7 nAChRs present on GABAergic, glutamatergic and cholinergic neurons can facilitate the release of neurotransmitters such as glutamate, acetylcholine and GABA whereas postsynaptic receptors can modulate other neuronal inputs and trigger a variety of down stream signaling pathways. Thus, α7 nAChRs may be important for cognitive functions involved in attention, learning, and memory. Support for this hypothesis has emerged from preclinical studies with selective agonists, antagonists, and more recently, positive allosteric modulators. α7 nAChR agonists such as PNU-282987, SSR180711A, AR-R17779 improve performance in rats in social recognition (Van Kampen et. al. Psychopharmacology Vol. 172, pages 375-383, 2004), maze training (Levin et. al. Behavioral pharmacology Vol. 10, pages 675-680, 1999; Arendash et. al. Brain research Vol. 674, pages 252-259, 1995) and active avoidance (Arendash et. al. Brain research Vol. 674, pages 252-259, 1995) models while α7 nAChR antagonists impair such performance (Bettany and Levin, Pharmacology Biochemistry and Behavior Vol. 70, pages 467-474, 2001; Felix and Levin, Neuroscience Vol. 81, pages 1009-1017, 1997). Both agonists and positive allosteric modulators, exemplified respectively by PNU-282987 and PNU-120596, have been shown to reverse auditory gating deficits in animal models (Martin et. al. Psychopharmacology Vol. 174, pages 54-64, 2004). The cognitive function of α7 nAChR may be mediated by calcium triggered signal transduction. Activation of α7 nAChR results in increased intracellular Ca2+, initiating a signal transduction cascade involving the activation of a variety of protein kinases and other proteins by phosphorylation, which ultimately leads to regulation of gene expression. The proteins that are phosphorylated in response to α7 nAChR activation include extracellular signal-regulated kinase 1/2 (ERK1/2) (Ren et. al. J. Neurochem Vol. 94, pages 926-933, 2005), cAMP response element binding protein (CREB) (Roman et. al. FASEB Vol. 18, pages 1436-143, 2004), and Akt (Shaw et. al. J. biol. chem. Vol. 277, pages 44920-44924, 2002).

α7nAChR has been implemented in neuroprotection. Nicotine and α7nACh can prevent neurons from cell death induced by growth factor withdrawal (Jonnala and Buccafusco, J Neurosci Res. Vol. 66, pages 565-72, 2001) or glutamate exposure (Donnelly-Roberts et. al. Brain Res. Vol. 719, pages 36-44, 1996) in an α7nAChR-dependent manner. α7nAChR has also been observed to interfere APP processing. Nicotine or α7nAChR agonists can increase sAPPα (Kim et. al. Mol Pharmacol. Vol. 52, pages 430-6, 1997; Xiu et. al. J Neurosci Res. Vol. 82, pages 531-41, 2003) in cell cultures. Administration of nicotine can reduce detergent-insoluble forms of Aβ₁₋₄₀ and Aβ₁₋₄₂ in APP transgenic mice (Hellstrom-Lindahl et. al. Eur J Neurosci. Vol. 19, pages 2703-10, 2004; Unger et. al. J Pharmacol Exp Ther. Vol. 317, pages 30-36, 2006) although the nAChRsubtype involved is unclear. All these observations suggest that α7n nAChR agonist may modify AD pathological pathway by interfering either with the toxicity or the production of amyloid β.

The present application provides for compounds of formula (I), as indicated below which are α7 nAChR selective ligands, and are capable of reducing Aβ production in the conditioned media of cell cultures,

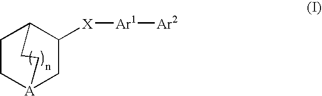

wherein:

n is 0, 1, or 2;

A is N or N⁺—O⁻;

X is selected from the group consisting of O, S, and —N(R¹)—;

Ar¹ is a 6-membered aromatic ring containing 0, 1, 2, 3, or 4 nitrogen atoms, wherein Ar¹ is substituted with 0, 1, 2, 3, or 4 alkyl groups;

Ar² is a group of the formula:

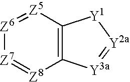

Z⁵, Z⁶, Z⁷, and Z⁸ are independently selected from the group consisting of C and —C(R^{3b}); provided that zero or one of Z⁵, Z⁶, Z⁷, and Z⁸ is C;

Y¹ at each occurrence is independently selected from the group consisting of O, S, —N(R²), —C(R³) , and —C(R³)(R^{3a});

Y^{2a} and Y^{3a} are independently selected from the group consisting of N, C and —C(R^{3a}); provided that when Y¹ is —C(R³) in a group of formula (b), Y^{2a} and Y^{3a} are selected from the group consisting of N and —C(R^{3a}), and when one of Y^{2a} and Y^{3a} is C, then Y¹ in a group of formula (b) is O, S, —N(R²), or —C (R³) (R^{3a});

wherein when one of Z⁵, Z⁶, Z⁷, and Z⁸ is C, then Y¹ in a group of formula (b) is selected from the group consisting of O, S, —N(R²) , and —C(R³)(R^{3a}); Y^{2a} and Y^{3a} are each independently selected from the group consisting of N and —C(R^{3a}); and the group of formula (b) is attached to Ar¹ through the C of Z⁵, Z⁶ Z⁷, or Z⁸ ; and also wherein when Y¹ in a group of formula (b) is —C(R³) or one of Y^{2a} and Y^{3a} is C, then Z⁵, Z⁶, Z⁷, and Z⁸ are each —C(R^{3b}) and the group of formula (b) is attached to Ar¹ through the C atom of —C(R³) of Y¹ in the group of formula (b) or through the C atom of Y^{2a} or Y^{3a};

R¹ and R² at each occurrence are each independently selected from the group consisting of hydrogen and alkyl;

R³ and R^{3a} at each occurrence are each independently selected from the group consisting of hydrogen, halogen, alkyl, aryl, —OR⁴, —NR⁵R⁶, -alkyl-OR⁴, and -alkyl-NR⁵R⁶;

R⁴ is selected from the group consisting of hydrogen, alkyl, aryl, alkylcarbonyl, and arylcarbonyl;

R⁵ and R⁶ at each occurrence are each independently selected from the group consisting of hydrogen, alkyl, aryl, alkylcarbonyl, alkoxycarbonyl, aryloxycarbonyl, and arylcarbonyl, provided that at least one of R⁵ and R⁶ is hydrogen or alkyl; and

R⁸ is selected from the group consisting of hydrogen and alkyl. Specific examples of these compounds are indicated in the examples of the present application.

The present application provides for α7 nAChR selective ligands of formula (I) that can be used to reduce Aβ production in the conditioned media of cell cultures, and therefore may be useful to reduce Aβ production in a patient suffering from a disorder involving an increase in Aβ formation.

The present application also provides for an immunoassay kit to be used for the in vitro quantitative determination of rodent amyloid β peptide, either Aβ₁₋₄₀ or Aβ₁₋₄₂, in a sample contacting the sample with a monoclonal antibody selective for N-temrinal of rodent Aβ for a time and under conditions to form complexes; contacting said complexes with a polyclonal anti-Aβ₁₋₄₀ or Aβ₁₋₄₂ antibody for a time and under conditions to form complexes; contacting said complexes with a secondary antibody linked to a detectable label capable of generating a measurable signal; and detecting the measurable signal which indicates the amount of rodent Aβ₁₋₄₀ or Aβ₁₋₄₂ in the sample.

It is intended that the rodent amyloid β peptide(s) in the sample selected from Aβ₁₋₄₀ and Aβ₁₋₄₂ are captured with solid-phase antibody carriers having said antibodies immobilized thereon.

The immunoassay kit described in the present application can be used for a sample selected from the group comprising brain homogenates, whole blood, serum, plasma, cerebrospinal fluid, urine, lymph fluids, and cell culture supernatants. It is contemplated within the scope of the present description that the immunoassay kit can be designed in a way that all steps are performed in less than 5 hours, preferably 3 hours, more preferably 2 hours.

The present application also discloses a method of detecting the presence of rodent amyloid β peptide(s) in a sample containing said amyloid β peptide(s), then contacting said sample with the immunoassay kit described above, and measuring the absence or presence of an interaction with the antibodies selective for the rodent amyloid β peptide in said immunoassay kit.

### EXAMPLES

The present application will now be described by way of examples, which are meant to illustrate, but not to limit, the scope of the subject matter described in the present specification.

### Example 1

### Assay for Detection of Mouse and Rat Aβ1-40 and Aβ1-42

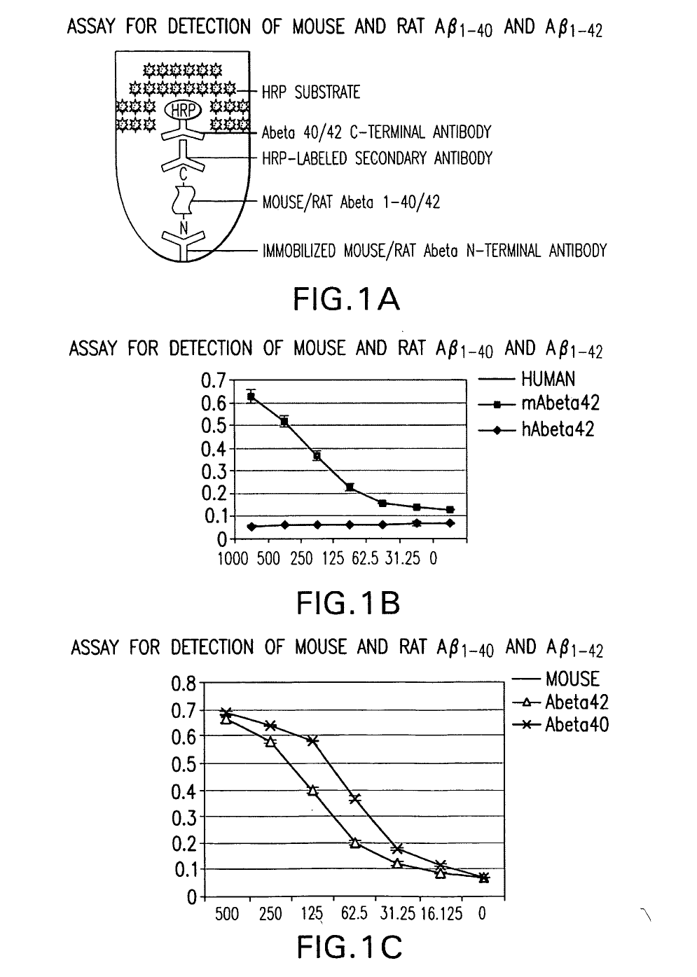

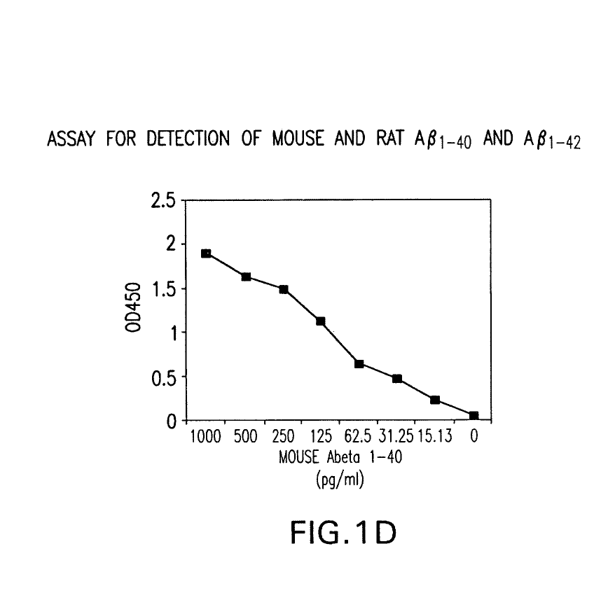

Mouse monoclonal antibody specific for the N-terminal of mouse/rat Aβ was immobilized by incubation of 96-well polystyrene plate with 50 ul of the antibody at 4 ug/ml in each well in a coating buffer (0.1 M NaHCO3 pH 8.2) overnight at 4° C. The plates were washed thereafter each step for 3 times with 200 μl/well PBST (PBS with 0.5% Tween-20). The wells were then sequentially incubated with 50 μl/well test samples containing mouse/rat or human Aβ1-40 or Aβ1-42 overnight at 4° C. Primary rabbit antibodies specific for either Aβ1-40 or Aβ1-42 (BioSource International Inc., Camarillo, Calif.) were added to each well at 50 ul/well for a final concentration of 400 ng/ml, and incubated at room temperature. After one hour, 50 μl/well horseradish peroxidase (HRP)-conjugated goat antibody specific for rabbit IgG (Jackson ImmunoResearch Latoratories, Inc., West Grove, Pa.) were added to each well and further incubated for 1 hour at room temperature. This step was followed by incubation of 50 ul/well HRP substrate 3,3′,5,5′-tetramethylbenzidine (TMB) for 30 minutes at room temperature. The colorimetric reaction was stopped by addition of 50 μl/well of 0.2 M sulfuric acid. The color signal was measured at 450 nM in a spectrophotometer. As shown in FIG. 1B, the assay detected both mouse/rat Aβ1-42 in a concentration-dependent manner and did not detect human Aβ1-42. This indicates the specificity of the assay for rodent Aβ The assay detected both mouse/rat Aβ₁₋₄₀ and Aβ₁₋₄₂ at levels less than 15 ng/ml (FIG. 1C).

Alternatively, the assay was performed in a short format with a single incubation: each of the antibody-coated wells described above was sequentially added with 25 μl samples and 25 μl antibody mixture containing 800 ng/ml primary rabbit antibodies and 400 ng/ml HRP-conjugated goat antibody specific for rabbit IgG, followed by the colorimetric reaction. The whole procedure can be completed in two hours. As shown in FIG. 1D, the sensitivity of the one-incubation assay for Aβ was less than 16 ng/ml.

### Example 2

### Assay for Detection of Endogenous Mouse and Rat Aβ1-40 and Aβ1-42 in Cell Culture Models, Animal Plasma and Brain Tissues

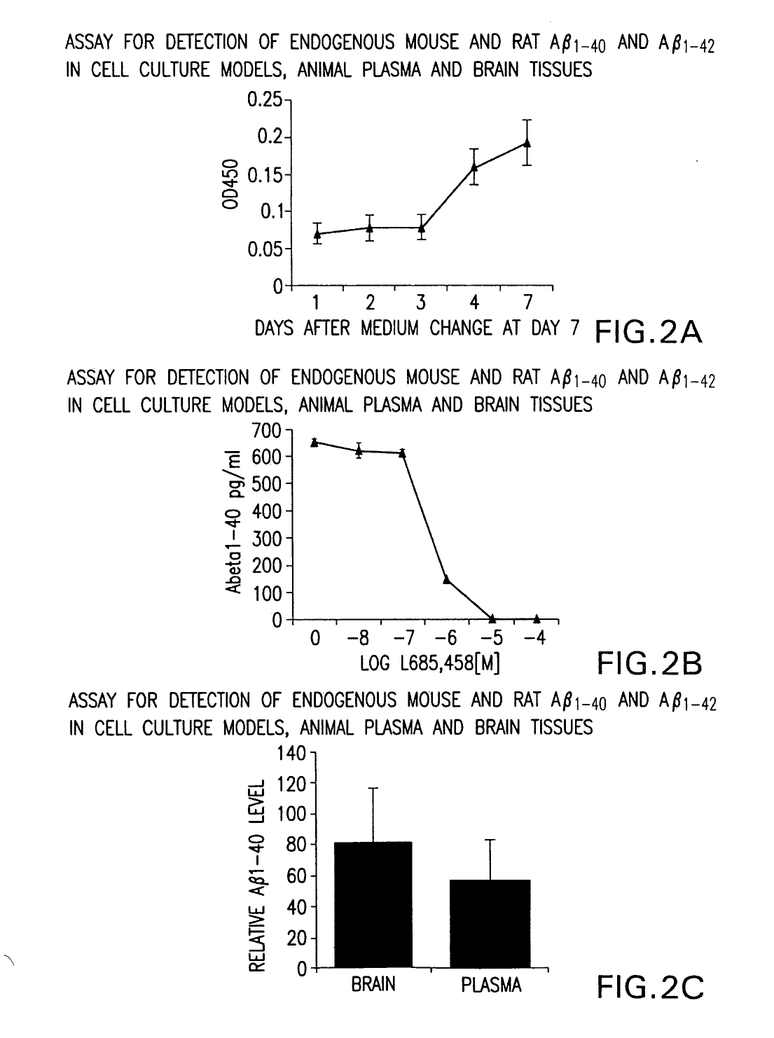

This example shows that the assay of the present invention detects and quantifies endogenous Aβ₁₋₄₀ released by rat cortical neurons. The amount released can be reduced by a γ-secretase inhibitor. Cortical neurons from brains of rats at postnatal day 0 was cultured in B27 medium (Invitrogen Corp., Carlsbad, Calif.) for 7 days then the medium was changed and the conditioned medium was collected thereafter at different days and tested in the assay as desribed in Example 1 at 1:2 dilution. FIG. 2A shows that Aβ₁₋₄₀ was detectable at day 4 after medium change and reached peak at day 7. The cortical neurons of 7 days of culture were treated with gamma-secretase inhibitor L-685,458 at various concentrations for 7 days and the Aβ₁₋₄₀ level was measured in the conditioned medium. The Aβ₁₋₄₀ level was reduced by the treatment in a concentration dependent manner with an IC₅₀ equal to 431 nM (FIG. 2B). L-685,458 (an aspartyl protease transition state mimic) is a potent inhibitor of gamma-secretase activity therefore can inhibit the production of Aβ by blocking the gamma-cleavage of APP. This result validated the cell model for measurement of compound effect on APP processing and Aβ₁₋₄₀ production. Aβ₁₋₄₀ was also detectable in samples of mouse brain and plasma, as shown in FIG. 2C. Mouse brain was homogenized in 5 M guanidine-HCl by sonication for 5 seconds in ice, followed by incubation for 3-4 hours at room temperature then centrifugation at 14,000 rpm for 20 minutes at 4C. The supernatant was saved for the assay. Mouse plasma was tested directly in the assay.

### Example 3

### High Throughput Assay for Screening Compounds that Modify Endogenous Production of Mouse and Rat Aβ₁₄₀ and Aβ₁₋₄₂

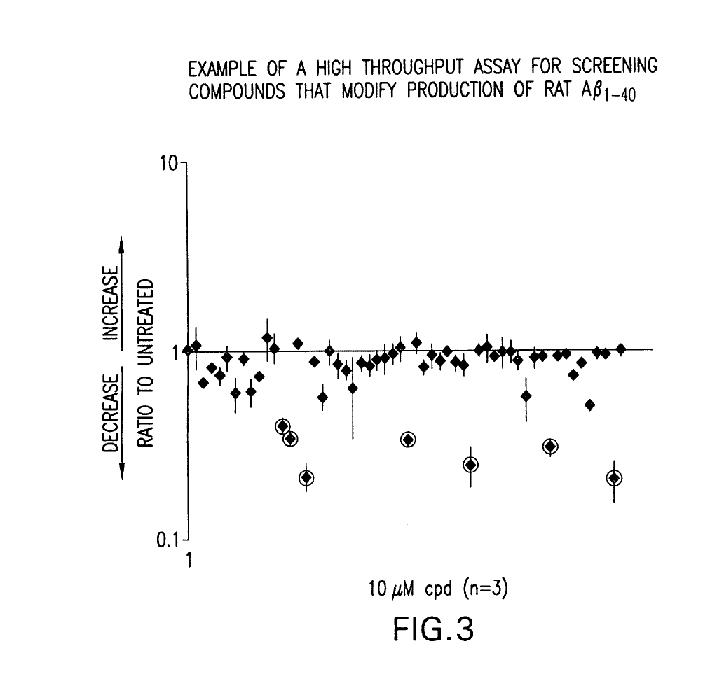

A collection of compounds was screened for inhibitors of Aβ production using the cortical neurons model. Cortical neurons were treated with compounds at a single concentration of 10 μM for 4 days, and Aβ levels in conditioned medium was measured as described in Example 2. FIG. 3 exemplifies a screening result. Assays were conducted in a 96-well format. Data points are mean value of Aβ₁₋₄₀ (ratio to untreated) of indicated compounds from a representative chemical library. The circled data points show compounds that reduce Aβ₁₋₄₀ more than 50%. Some of the identified compounds are ligands of the nicotinic receptor, particularly α7 nAChR.

### Example 4

### Identification of Compounds such as Nicotinic Acetylcholine Receptor Ligands

The effect of nicotinic receptor ligands on Aβ production was analyzed by measurement of the Aβ₁₋₄₀ level in the conditioned media of rat cortical cultures treated with α7 nAChR selective ligands for 7 days. The tested compounds, α7 ligands, were: Compound A, N-[(3R)-1-Azabicyclo[2.2.2]oct-3-yl]-4-chlorobenzamide hydrochloride; Compound B, (R)-3-(5-(1H-indol-5-yl)-pyrimidin-2-yloxy)-1-aza-bicyclo[2.2.2]octane; and Compound C, (R)-3-[6-(1H-Inden-5-yl)-pyridazin-3-yloxy]-1-aza-bicyclo[2.2.2]octane.

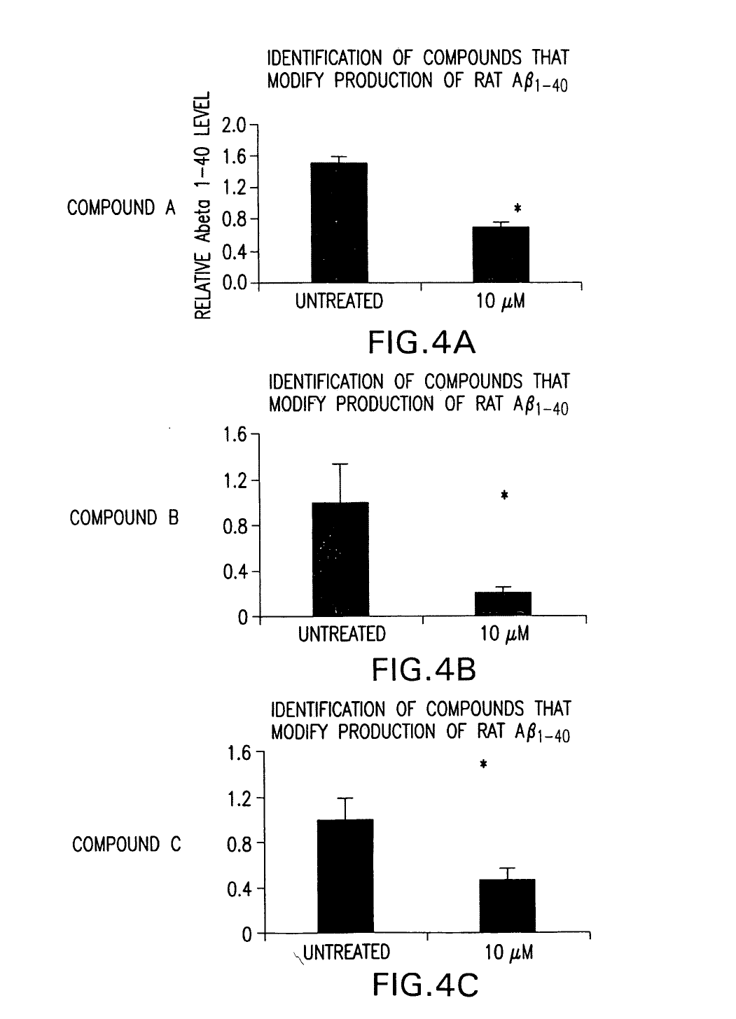

The treatment with these structurally diverse compounds reduced Aβ₁₋₄₀ level by 30-50% at 10-100 μM (FIG. 4). The mechanism underlying the effect of nAChR ligands on Aβ production could include, but may not be limited to: (1) Increased α-secretase cleavage, as suggested by the observed increase in sAPP in Nicotine treated cell cultures (Kim et. Al. Mol. Pharm. 52:430, 1997; Hellstron-Lindahl et. Al. Eur. J. Neurosci. 19:2703, 2004) and rats (Utiski et. Al. J. Alz. Dis. Vol. 4 page 405, 200), which in turn reduced substrate for β- and γ-secretase for Aβ production; (2) Down-regulation of APP expression; (3) Modification of APP/Aβ transportation.

What is claimed is:

1. An immunoassay method for detecting the presence and measuring the amount of rodent amyloid β peptides in a sample without cross-reacting with human Aβ₁₋₄₂, comprising the steps of:
a. contacting the sample with a monoclonal antibody selective for N-temrinal of rodent Aβ for a time and under conditions to form complexes;
b. contacting said complexes with a polyclonal anti-Aβ₁₋₄₀ or Aβ₁₋₄₂ antibody for a time and under conditions to form complexes;
c. contacting said complexes with a secondary antibody linked to a detectable label capable of generating a measurable signal;
d. incubating the mixture of step (c) for a time and under conditions to form complexes and to develop a measurable signal; and
e. determining the amount of amyloid β peptide in the sample by detecting and measuring the signal generated.

2. The method of claim 1, wherein the rodent amyloid β peptide is selected from the group comprising Aβ₁₋₄₀ and Aβ₁₋₄₂.

3. The method of claim 1 wherein the sample is selected from the group comprising brain homogenates, whole blood, serum, plasma, cerebrospinal fluid, urine, lymph fluids, and cell culture supernatants.

4. The method of claim 1 wherein the sample size is in the range of 1-25 μL.

5. The method of claim 1 wherein steps (a)-(e) are performed in less than 5 hours, preferably, 3 hours, more preferably 2 hours.

6. The method of claim 1 wherein the antibodies used in step (a) are attached to a solid phase.

7. The method of claim 1, wherein steps (a), (b), (c), (d) and (e) are performed at the same time in one step.

8. A method for identifying an agent that alters the production of endogenous rodent amyloid β peptide in a primary neuron cell culture.

9. The method of claim 8, comprising the steps of:
i. contacting the agent with the rodent primary neuron cell culture;
ii. detecting the production of amyloid β peptide in the culture in the presence or absence of the agent using the method of claim 1, and
iii. comparing the amounts of detected amyloid β peptide in the presence or absence of the agent, wherein said comparison identifies the agent as an agent that alters the production of amyloid β peptide.

10. The method of claim 8, wherein the endogenous rodent amyloid β peptide is selected from the group comprising Aβ₁₋₄₀ and Aβ₁₋₄₂.

11. An immunoassay kit to be used for the in vitro quantitative determination of rodent amyloid β peptide in a sample without cross-reacting with human Aβ1-42, comprising the steps of (a) contacting the sample containing the rodent amyloid β peptide(s) with a monoclonal antibody selective for the rodent N-terminal of mouse/rat Aβ for a time and under conditions to form complexes; (b) contacting said complexes with a polyclonal anti-Aβ₁₋₄₀ or Aβ₁₋₄₂ antibody for a time and under conditions to form complexes; (c) contacting said complexes with a secondary antibody linked to a detectable label capable of generating a measurable signal; and (d) detecting the measurable signal which indicates the amount of rodent amyloid β peptide in the sample.

12. The immunoassay kit of claim 11 wherein the rodent amyloid β peptide in the sample is selected from Aβ₁₋₄₀ and

13. The immunoassay kit of claim 11, wherein the sample is selected from the group comprising brain homogenates, whole blood, serum, plasma, cerebrospinal fluid, urine, lymph fluids, and cell culture supernatants.

14. The immunoassay kit of claim 11, wherein the rodent amyloid Peptide in the sample selected from Aβ₁₋₄₀ and Aβ₁₋₄₂ are captured with solid-phase antibody carriers having said antibodies immobilized thereon.

15. The immunoassay kit of claim 11, wherein steps (a)-(d) are performed in less than 5 hours, preferably 3 hours, more preferably 2 hours.

16. The immunoassay kit of claim 11, wherein steps (a)-(d) are performed at the same time in one step.

17. A method of detecting the presence of rodent amyloid β peptide, comprising obtaining a sample comprising a rodent amyloid β peptide; contacting said sample with the immunoassay kit according to claim 10; and measuring the absence or presence of an interaction with the antibodies selective for the rodent amyloid β peptide in said immunoassay kit.

18. A compound of formula (I), 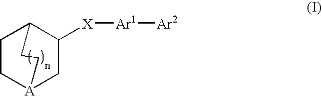
wherein:
n is 0, 1, or 2;
A is N or N⁺—O⁻;
X is selected from the group consisting of O, S, and —N(R¹)—;
Ar¹ is a 6-membered aromatic ring containing 0, 1, 2, 3, or 4 nitrogen atoms, wherein Ar¹ is substituted with 0, 1, 2, 3, or 4 alkyl groups;
Ar² is a group of the formula: 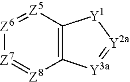
Z⁵, Z⁶, Z⁷, and Z⁸ are independently selected from the group consisting of C and —C(R^{3b}); provided that zero or one of Z⁵, Z⁶, Z⁷, and Z⁸ is C;
Y¹ at each occurrence is independently selected from the group consisting of O, S, —N(R²), —C(R³), and —C(R³) (R^{3a});
Y^{2a} and Y^{3a} are independently selected from the group consisting of N, C and —C(R^{3a}); provided that when Y¹ is —C(R³) in a group of formula (b), Y^{2a} and Y^{3a} are selected from the group consisting of N and —C(R^{3a}), and when one of Y^{2a} and Y^{3a} is C, then Y¹ in a group of formula (b) is O, S, —N(R²), or —C(R³)(R^{3a});
wherein when one of Z⁵, Z⁶, Z⁷, and Z⁸ is C, then Y¹ in a group of formula (b) is selected from the group consisting of O, S, —N(R²) and —C(R³)(R^{3a}); Y^{2a} and Y^{3a} are each independently selected from the group consisting of N and —C(R^{3a}); and the group of formula (b) is attached to Ar¹ through the C of Z⁵Z⁶, Z⁷, or Z⁸; and also wherein when Y¹ in a group of formula (b) is —C(R³) or one of Y^{2a} and Y^{3a} is C, then Z⁵, Z⁶, Z⁷, and Z⁸ are each —C(R^{3b}) and the group of formula (b) is attached to Ar¹ through the C atom of —C(R³) of Y¹ in the group of formula (b) or through the C atom of Y^{2a} or Y^{3a};
R¹ and R² at each occurrence are each independently selected from the group consisting of hydrogen and alkyl;
R³ and R^{3a} at each occurrence are each independently selected from the group consisting of hydrogen, halogen, alkyl, aryl, —OR⁴, —NR⁵R⁶, -alkyl-OR⁴, and -alkyl-NR⁵R⁶;
R⁴ is selected from the group consisting of hydrogen, alkyl, aryl, alkylcarbonyl, and arylcarbonyl;
R⁵ and R⁶ at each occurrence are each independently selected from the group consisting of hydrogen, alkyl, aryl, alkylcarbonyl, alkoxycarbonyl, aryloxycarbonyl, and arylcarbonyl, provided that at least one of R⁵ and R⁶ is hydrogen or alkyl; and
R⁸ is selected from the group consisting of hydrogen and alkyl; and which is an α7 nAChR selective ligand, that is capable of reducing Aβ production in the conditioned media of cell cultures.

19. The compound of claim 18, comprising
(R)-3-(5-(1H-indol-5-yl)-pyrimidin-2-yloxy)-1-aza-bicyclo[2.2.2]octane; and
(R)-3-[6-(1H-Inden-5-yl)-pyridazin-3-yloxy]-1-aza-bicyclo[2.2.2]octane.

20. The use of α7 nAChR selective ligands to reduce Aβ production in the conditioned media of cell cultures.

21. The use of α7 nAChR selective ligands to reduce Aβ production in a patient suffering from a disorder involving an increase in Aβ formation.
